(19) United States
(12) Patent Application Publication
(10) Pub. No.: US 2013/0197956 A1
(43) Pub. Date: Aug. 1, 2013

(54) COMPUTER-IMPLEMENTED METHOD, SYSTEM, AND COMPUTER PROGRAM FOR SCHEDULING INTERPRETERS

(71) Applicants: Dennis Ayzin, Overland Park, KS (US); Mira Mdivani, Overland Park, KS (US)

(72) Inventors: Dennis Ayzin, Overland Park, KS (US); Mira Mdivani, Overland Park, KS (US)

(21) Appl. No.: 13/364,090

(22) Filed: Feb. 1, 2012

(51) Int. Cl. G06Q 10/06

(52) U.S. Cl. 705/7.14

(57) ABSTRACT

A computer-implemented method, system, and computer program for scheduling interpreters. The method includes receiving at a computer system information from a number of interpreters; storing at least some of the interpreter information in memory accessible by the computer system; receiving at the computer system a request from a customer for an interpreter to perform an interpretation assignment; searching with the computer system the interpreter information in the memory to identify interpreters who meet a criteria of the request; providing information about the interpretation assignment to the interpreters who meet the criteria; receiving at the computer system an availability acknowledgment from at least one of the interpreters; scheduling the interpretation assignment with one of the interpreters who provides the availability acknowledgment; and notifying the customer that the interpretation assignment has been scheduled.

### BACKGROUND

Many people residing in or visiting the United States do not speak English well enough to provide information to and/or receive information from others. Language barriers are especially problematic for people seeking medical care or dealing with legal issues because communication problems in these circumstances can lead to dire consequences. Interpreters are therefore often hired by hospitals, doctors, and emergency responders to facilitate communication with non-English speaking patients and/or their guardians and by courts, law firm, and lawyers to facilitate communications with suspects, witnesses, and/or parties to lawsuits and other disputes. Interpreters are also often used for many other circumstances.

Identifying and scheduling qualified interpreters can be very time-consuming because the interpreters must have language skills that precisely match the languages spoken by the parties and must also be nearby and available on specific days and times. Many translations and interpreting service companies have been formed to assist with the locating and scheduling of interpreters. However, even professional translation and interpreting service companies often have difficulty in quickly and efficiently identifying and scheduling interpreters and then tracking the completion of the interpretation assignments.

### SUMMARY

The present invention solves the above-described problems and provides a distinct advance in the art of interpreter scheduling services. More particularly, the present invention provides a computer-implemented method, system, and computer program for identifying and scheduling qualified interpreters for interpretation assignments and for tracking the projects through completion.

An embodiment of the invention may be implemented with a computer system operated by or for an interpretation or translation scheduling company, an interpreting department at a medical provider or court system, or other entity that provides interpreters for interpretation assignments. The computer system may be used to implement a method that identifies and schedules qualified interpreters for interpretation assignments and tracks the assignments through completion. In one embodiment, the computer-implemented method comprises some of the following steps: receiving information from a number of interpreters; storing at least some of the interpreter information in memory accessible by the computer system; receiving a request from a customer for an interpreter to perform an interpretation assignment; searching the interpreter information in the memory to identify interpreters who meet a criteria of the request; providing information about the interpretation assignment to the interpreters who meet the criteria; receiving an availability acknowledgment from at least one of the interpreters; scheduling the interpretation assignment with one of the interpreters; and notifying the customer who requested the interpreter that the interpretation assignment has been scheduled.

In other embodiments, the computer-implemented method also comprises some of the following steps: providing a calendar accessible on the computer system; displaying a calendar entry for an interpretation assignment; and linking the calendar entry to at least some of the interpreter information for the interpreter who is scheduled to handle the interpretation assignment. The computer-implemented method may further comprise the steps of: providing an interpretation agreement to the interpreter who is scheduled to handle the interpretation assignment; storing an executed copy of the interpretation assignment in memory accessible by the computer system; and linking the interpretation agreement with the calendar entry for the assignment. The computer-implemented method further comprise the steps of: receiving from the interpreter a time sheet after the interpretation assignment is complete; storing an executed copy of the time sheet in memory accessible by the computer system; and linking the time sheet with the calendar entry for the assignment.

This summary is provided to introduce a selection of concepts in a simplified form that are further described in the detailed description below. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 is a block diagram that illustrates persons and/or entities that may be involved with aspects of the present invention.

FIG. 2 is a schematic diagram of exemplary computer and communications equipment that may be used to implement embodiments of the invention.

FIG. 3 is a flow diagram illustrating steps in a method of the invention and/or code segments in a computer program of the invention.

FIG. 4 is an exemplary screen display that may be presented by one or more computer programs of the present invention.

FIG. 5 is another exemplary screen display that may be presented by the computer programs of the present invention.

FIG. 6 is another exemplary screen display that may be presented by the computer programs of the present invention.

FIG. 7 is another exemplary screen display that may be presented by the computer programs of the present invention.

FIG. 8 is another exemplary screen display that may be presented by the computer programs of the present invention.

FIG. 9 is another exemplary screen display that may be presented by the computer programs of the present invention.

FIG. 10 is another exemplary screen display that may be presented by the computer programs of the present invention.

FIG. 11 is another exemplary screen display that may be presented by the computer programs of the present invention.

FIG. 12 is another exemplary screen display that may be presented by the computer programs of the present invention.

FIG. 13 is another exemplary screen display that may be presented by the computer programs of the present invention.

FIG. 14 is another exemplary screen display that may be presented by the computer programs of the present invention.

FIG. 15 is another exemplary screen display that may be presented by the computer programs of the present invention.

The drawing figures do not limit the present invention to the specific embodiments disclosed and described herein. The drawings are not necessarily to scale, emphasis instead being placed upon clearly illustrating the principles of the invention.

### DETAILED DESCRIPTION

The following detailed description of embodiments of the invention references the accompanying drawings. The embodiments are intended to describe aspects of the invention in sufficient detail to enable those skilled in the art to practice the invention. Other embodiments can be utilized and changes can be made without departing from the scope of the claims. The following detailed description is, therefore, not to be taken in a limiting sense. The scope of the present invention is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, act, etc. described in one embodiment may also be included in other embodiments, but is not necessarily included. Thus, the present technology can include a variety of combinations and/or integrations of the embodiments described herein.

The present invention provides various embodiments of a computer-implemented method, system, and computer program for identifying and scheduling qualified interpreters for interpretation assignments and for tracking the assignments through completion. As used herein, “interpretation assignment”may include any instance where two or more people who speak at least one different language must communicate. For example, an interpretation assignment may include a medical consultation between an English speaking doctor and a non-English speaking patient or a court deposition between an English speaking attorney and a non-English speaking witness. An interpretation assignment may also include a job interview, court hearing, meeting, etc.

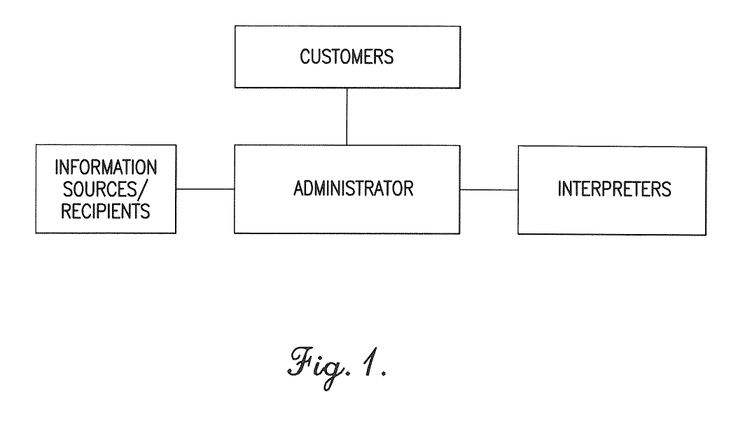

FIG. 1 is a block diagram that illustrates the relationships between persons and/or entities that may be involved in an interpretation assignment. The entities include an administrator, one or more customers, one or more interpreters, and possibly one or more information sources and/or recipients.

The administrator can be any entity or person that provides interpretation scheduling services or similar services. The administrator may be an independent organization or may be controlled by one of the customers. In one embodiment, the administrator may be an interpreter or translation scheduling company such as TranslationPerfect.com of Overland Park, Kans.

The interpreters may be any individuals that speak multiple languages and desire to provide interpretation services. The interpreters may be independent contractors or may be employees of the administrator or the customer. For example, the interpreters may be scheduled and hired by the administrator to provide interpretation services to the customers.

The customers may be hospitals, doctors, law firms, lawyers, or any other entities or persons that require interpretation services. The customers typically contract with the administrator to find interpreters for the customers' interpretation assignments.

The information sources/recipients may be any persons or entities that provide information to or receive information from the administrator or the customers. For example, the information sources/recipients may include employment agencies, governmental agencies, courts, hospitals etc.

In some embodiments the administrator, customers, and interpreters may be separate entities and in other embodiments they may be associated with one another. For example, employees of one of the customers can provide the services of the administrator and/or employees of the administrator may be the interpreters.

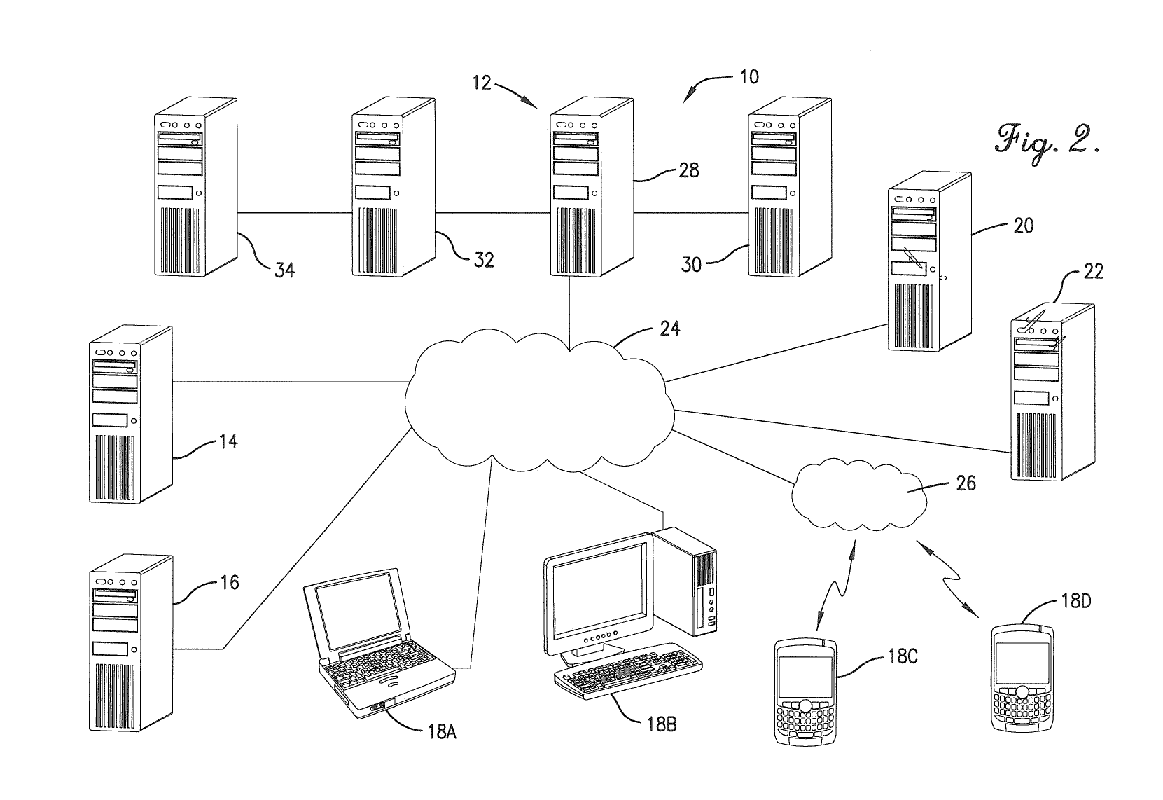

Aspects of the invention can be implemented with computer hardware, software, firmware, or a combination thereof. In one embodiment, aspects of the invention may be at least partially implemented with computer and communications equipment broadly referred to by the numeral 10 in FIG. 2. An embodiment of the computer and communications equipment 10 includes a computer system 12 that may be operated by or for the administrator, one or more computer systems 14, 16 that may be operated by the customers, one or more computers or portable electronic devices 18A, 18B, 18C, 18D that may be operated by interpreters, one or more computer systems 20, 22 that may be operated by information sources/recipients, a communications network 24, and a wireless telecommunications network 26. The components of the computer and communication equipment 10 illustrated and described herein are merely examples of equipment that may be used to implement embodiments of the present invention and may be replaced with other equipment without departing from the scope of the present invention.

In more detail, the computer system 12 stores or access data and other information that may be used by an administrator to schedule and track interpretation assignments. The computer system 12 may also implement one or more computer programs for performing some of the functions described herein. The computer system 12 may also provide a web-based portal that can be accessed by the administrator, customers, interpreters, and others.

Embodiments of the computer system 12 may include one or more servers running Windows; LAMP (Linux, Apache HTTP server, MySQL, and PHP/Perl/Python); Java; AJAX; NT; Novel Netware; Unix; or any other software system. The computer system 12 includes or has access to computer memory and other hardware and software for receiving, storing, accessing, and transmitting information as described below. The computer system 12 also includes conventional web hosting operating software, searching algorithms, an Internet connection, and is assigned a URL and corresponding domain name such as “translationperfect.com” so that it can be accessed via the Internet in a conventional manner.

One particular embodiment of the computer system 12 may comprise a web server 28, a database server 30, an application server 32, and/or an FTP server 34. However, the number and type of servers in the computer system 12 is a matter of design choice and may depend on the number of interpretation assignments handled by the administrator and the number of requests and other queries received by the computer system 12. Thus, the invention is not limited to the specific servers and other equipment described and illustrated herein.

The computer systems 14, 16 may be operated by one or more customers that wish to hire interpreters to handle an interpretation assignments. For example, the systems 14, 16 may be operated by hospitals, doctors, law firms, lawyers or other entities or persons. Each computer system 14, 16 may comprise any number of computers, servers or other equipment that provides data to and receives data from the computer system 12 via the communications network 24 or any other network. Although only two computer systems 14, 16 are shown in FIG. 2, it is understood that any number of computer systems operated by any number of customers may interact with the computer system 12.

The computing devices 18A-D may be any devices used by interpreters to access the computer system 12 via the communications network 24, the wireless network 26, and/or any other network. For example, the computing devices may be mobile phones or similar devices that can make and receive wireless communications such as phone calls, SMS texts, MMS messages, SMTP messages, etc. via the wireless telecommunication network 26. The computing devices may also include phone-enabled personal digital assistants, phone-enabled MP3 devices, phone-enabled handheld game players, phone-enabled tablet computers, or any other wireless communication devices. In some embodiments, the portable electronic devices are “smart” phones such as those manufactured by Apple®, Blackberry®, or Motorola®.

The computing devices may also be personal computers, laptop computers, tablet computers, handheld gaming devices or other portable personal computers. Each computing device 18A-D preferably includes or can access an Internet browser and a conventional Internet connection such as a wired or wireless broadband connection, modem, DSL converter, or ISDN converter so that it can access the computer system 12.

The computer systems 20, 22 may be operated by information sources or recipients such as employment agencies, courts, hospitals, etc. The computer systems 20, 22 may be any type of computers.

The communications network 24 is preferably the Internet but may be any other communications network such as a local area network, a wide area network, or an intranet. The wireless network 26 may be any network capable of supporting wireless communications such as the wireless networks operated by AT&T, Verizon, or Sprint. The wireless network may include conventional switching and routing equipment. The communications network 24 and wireless network 26 may also be combined or implemented with several different networks.

Embodiments of the present invention also comprise one or more computer programs stored in or on computer-readable medium residing on or accessible by the computer system 12, or other computer equipment. The computer programs may comprise listings of executable instructions for implementing logical functions in the computer equipment. The computer programs can be embodied in any non-transitory computer-readable medium for use by or in connection with an instruction execution system, apparatus, or device, such as a computer-based system, processor-containing system, or other system that can fetch the instructions from the instruction execution system, apparatus, or device, and execute the instructions. In the context of this application, a “computer-readable medium” can be any non-transitory means that can contain, store, or communicate the programs. The computer-readable medium can be, for example, but not limited to, an electronic, magnetic, optical, electro-magnetic, infrared, or semi-conductor system, apparatus, or device. More specific, although not inclusive, examples of the computer-readable medium would include the following: an electrical connection having one or more wires, a portable computer diskette, a random access memory (RAM), a read-only memory (ROM), an erasable, programmable, read-only memory (EPROM or Flash memory), an optical fiber, and a portable compact disk read-only memory (CDROM).

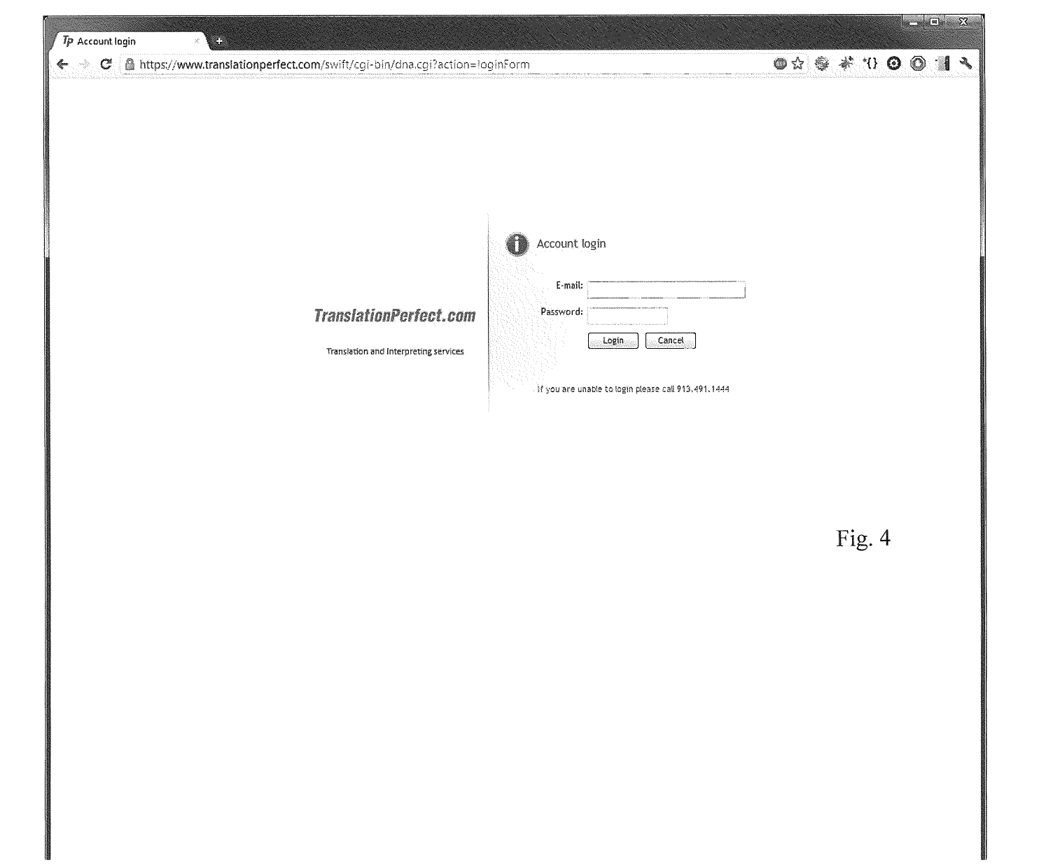

Aspects of the invention may be implemented with a website or other computer accessible site that is hosted by the computer system 12. The website or other site may support and display the screens of FIGS. 4-14 and other screens. The administrator, customers, and interpreters may access the site by entering the appropriate URL into a browser and then populating a log-in screen such as the one shown in FIG. 4. Users may be given access to particular portions of the website based on the log-in information. For example, administrators may be given access to all portions and pages of the site, whereas customers and interpreters may only be given access to certain portions or pages of the site that are relevant to them.

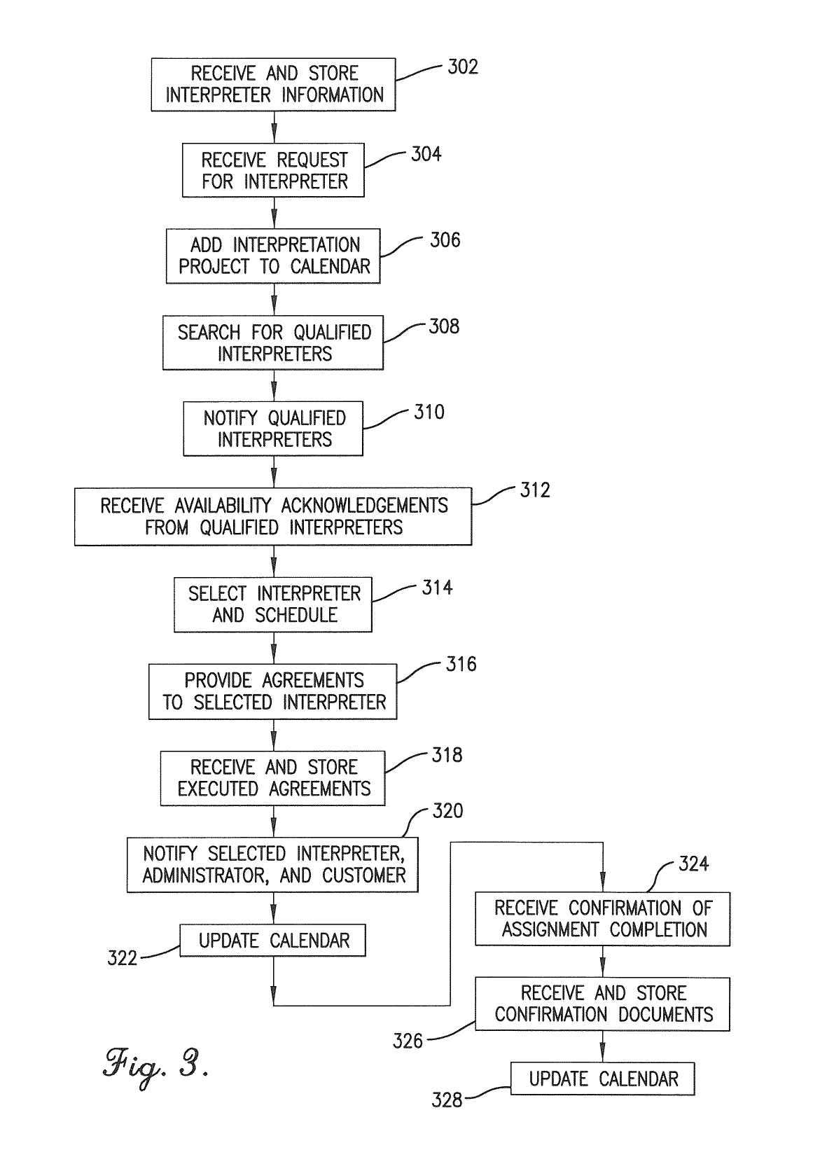

The flow chart of FIG. 3 shows the functionality and operation of exemplary embodiments of the present invention in more detail. In this regard, some of the blocks of the flow chart may represent method steps and/or module segments or portions of code of the computer programs of the present invention. The computer programs may comprise one or more executable instructions for implementing the specified logical function or functions. In some alternative implementations, the functions noted in the various blocks may occur out of the order depicted in FIG. 3. For example, two blocks shown in succession in FIG. 3 may in fact be executed substantially concurrently, or the blocks may sometimes be executed in the reverse order depending upon the functionality involved. Some of the module segments or code of the computer programs may display, populate, or otherwise enable the screen displays shown in FIGS. 4-14.

An embodiment of the invention may begin when information for a number of interpreters is received and stored in memory accessible by the computer system 12 as depicted in box 302 of FIG. 3. The information may include each interpreter's name, age, address, phone number, email address, and any other personal information needed for accounting, contract, employment, and compliance purposes. The information may also include a listing of all languages spoken by the interpreter as well as other professional information that can be used to match interpreters with interpretation assignments as explained in more detail below.

The interpreter information may be gathered in a variety of manners. For example, the information may be uploaded to the computer system 12 by the interpreters through an on-line interview process or may be manually entered by the administrator or other person.

In one embodiment, each interpreter enters and maintains a profile in an account store in or accessible by the computer system 12. Each profile may include the information mentioned above plus a list of professional certifications, rewards, honors, achievements, etc. and links to supporting documents such as resumes, college transcripts, background checks, vaccination records, letters of reference, etc. By requiring each interpreter to provide, update, and supplement their own information, the information is more likely to be as accurate and up-to-date as possible.

The interpreter information may be stored and categorized or otherwise classified by criteria that aids in the matching of interpreters with interpretation assignments. For example, the information my be tagged so that all interpreters with certain language skills can be quickly identified by the skill.

After at least some of the interpreter information is stored in the computer system 12, a request for an interpreter to handle an interpretation assignment may be received from a customer as shown in box 304. The request may include details of the interpretation assignment such as its location, date, and time; the languages to be interpreted; and details about the people being interpreted. The request, and any related information, may be sent to the administrator in any conventional manner and stored in the computer system 12. For example, the customer may access the computer system 12 directly and complete an on-line request, or the customer may call, fax, or email the administrator with an interpreter request and the administrator may enter the information into the computer system 12.

Once a request for an interpreter is received, details of the requested interpretation assignment are saved on the computer system 12. In one embodiment, details of the assignment may be added to a calendar supported by the computer system as depicted in box 306 and as described in more detail below.

After the request is received, one or more qualified interpreters must be identified for the interpretation assignment as depicted in box 308. This task may be done in a variety of manners. For example, the administrator may access the interpreter information stored in the computer system 12 and manually or automatically search for suitable interpreters or software on the computer system 12 may assist with the selection of qualified interpreters as described in more detail below.

Once one or more qualified interpreters are identified, they are notified of the interpretation assignment as shown in box 310. Again, this may be done in a variety of manners. For example, the administrator may simply call or e-mail the qualified interpreters or the computer system may automatically send notices to the qualified interpreters.

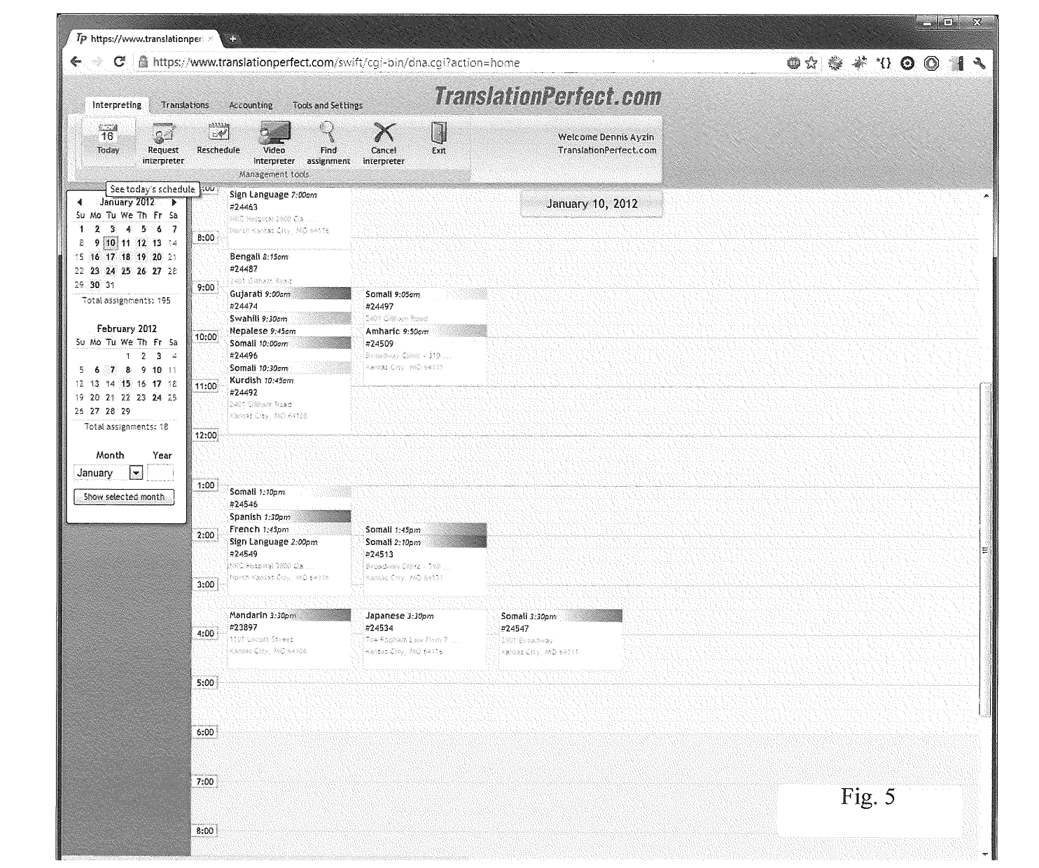

The screen shots of FIGS. 4-6 illustrate an exemplary on-line interpreter request and selection procedure which will now be described in more detail. A customer may initially use one of the computers 14, 16 or any other computers to access the computer system 12 and to log-in with the screen shown in FIG. 4. After log-in, the customer may be presented with the screen of FIG. 5 or a similar screen.

The screen of FIG. 5 includes a toolbar with a number of tabs including “Today”,

“Request Interpreter”, “Reschedule”, “Find Assignment”, and “Cancel Interpreter”. In one embodiment, the screen of FIG. 5 defaults to “Today” and displays several months of a calendar and a detailed daily calendar. As explained in more detail below, interpretation assignments may be added as entries on the calendar.

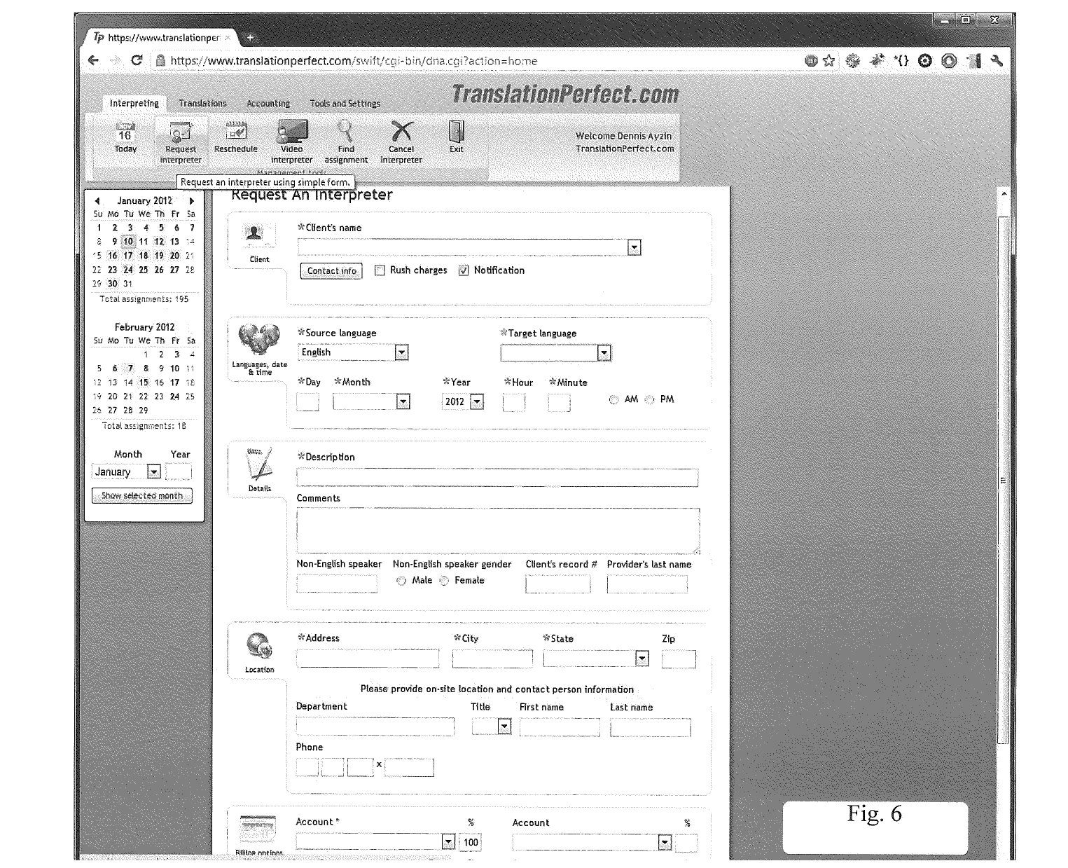

To initiate an interpreter request, the customer may press the “Request Interpreter” tab or button on the screen of FIG. 5 and be presented with the screen of FIG. 6. The customer may enter the customer's name (or this may be pre-populated based on the log-in), the source and target languages for the interpretation assignment, the date for the interpretation assignment, a description of the interpretation assignment, the name of the non-English speaking person to be interpreted, the address for the interpretation assignment, and account or billing information for the project. Several accounts may be listed to divide the costs of an interpretation assignment between several parties.

Once the screen of FIG. 6 has been populated, the customer may press a “Submit” button or similar button. This triggers the computer system 12 to perform a number of activities, including sending confirmation messages to the administrator and the customer, adding the requested interpretation assignment as an entry on the calendar in FIG. 5, and identifying and contacting qualified interpreters who may be able to handle the interpretation.

In one embodiment, the computer system identifies qualified interpreters by accessing the interpreter information stored in memory and searching for interpreters who match criteria of the request. For example, a search algorithm may identify interpreters that are within a specified distance (e.g. 20 miles) of the location for the interpretation assignment and interpreters who possess language skills, professional certifications, and/or other experience that matches certain criteria listed on the interpreter request. The administrator may tailor the search to prioritize or rank the search criteria. For example, particular language skills may be a necessary criteria whereas other criteria such as distance from the assignment, age of interpreter, etc. may be optional.

Once the computer system 12 identifies one or more qualified interpreters, it notifies them of the assignment as depicted in box 310. In one embodiment, the computer system broadcasts a summary of the assignment, such as its date, location, languages to be interpreted, etc. to the qualified interpreters. Each qualified interpreter may then view the summary of the assignment with one of the computing devices 18A-D and indicate whether they are available to handle it. In one embodiment, the available interpreters login to the computer system 12 and provide an indication of their availability as depicted by box 312. In other embodiments, the available interpreters may simply call or email the administrator to indicate their availability.

One or more of the available interpreters must then be selected for the interpretation assignment as depicted in box 314. The interpreters may be selected in a variety of manners. In one embodiment, the computer system 12 receives the availability messages from the interpreters and presents a list of the available interpreters to the administrator. The administrator may then manually or automatically select which of the interpreters to assign to a project and may then notify them via email or other means. In another embodiment, the first interpreter, or the first specified number of interpreters if more than one is required, who indicate availability for the interpretation assignment are automatically chosen.

The computer system 12 also sends agreements to the selected interpreters that must be executed as depicted in box 316. Alternatively, the computer system may send or provide a link to the agreements. Once executed and uploaded to the computer system, the agreements are stored in memory accessible by the computer system as depicted in box 318.

Once an interpreter has been selected and before or after the interpreter completes and submits all the necessary paperwork, the computer system 12 sends a confirmation message to the interpreter, the administrator, and the customer to confirm that the interpretation assignment has been scheduled as depicted in box 320. The computer system also updates the calendar to indicate that the interpretation assignment is now staffed with an interpreter as depicted in box 322.

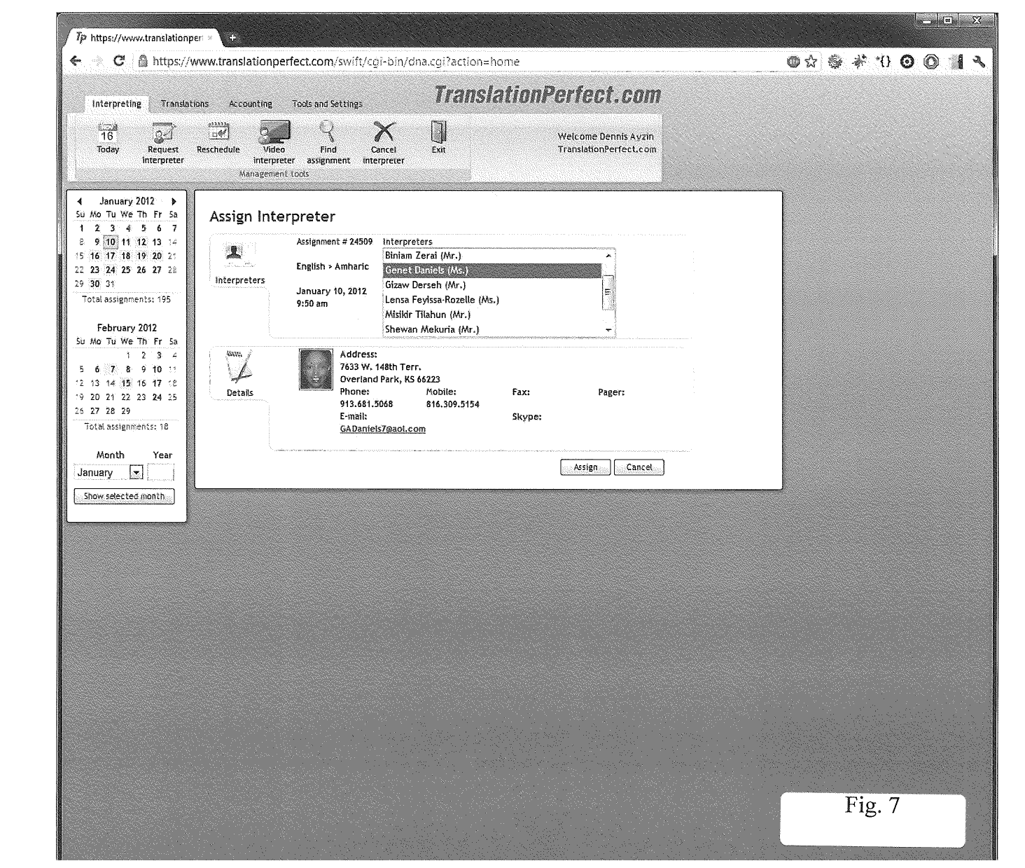

The screen shot shown in FIG. 7 illustrates an exemplary procedure for selecting an interpreter for a project which will now be described in more detail. The screen may be presented when an administrator selects calendar entry from FIG. 5 as described below. The Assign Interpreter screen may display details of the interpretation assignment along side a list of the qualified interpreters who indicated they were available for the assignment. In one embodiment, the interpreters are listed on the screen in the order they responded to the assignment notice so that interpreters who respond quickly are listed at the top of the list.

When the administrator selects one of the interpreters from the list, a photo of the interpreter and other information about the interpreter may be displayed as shown in FIG. 7. After reviewing information for one or more interpreters, the administrator may select one of the interpreters for an assignment by highlighting the interpreter's name in the list and then selecting an “Assign” button.

Clicking the “Assign” button in FIG. 7 prompts the computer system to perform a number of activities, including adding the selected interpreter's name, photo, and other information to the calendar entry for the interpretation assignment, changing the status of the interpretation assignment from unassigned to assigned, and sending confirmation messages via email or other means to the selected interpreter, the administrator, and the customer.

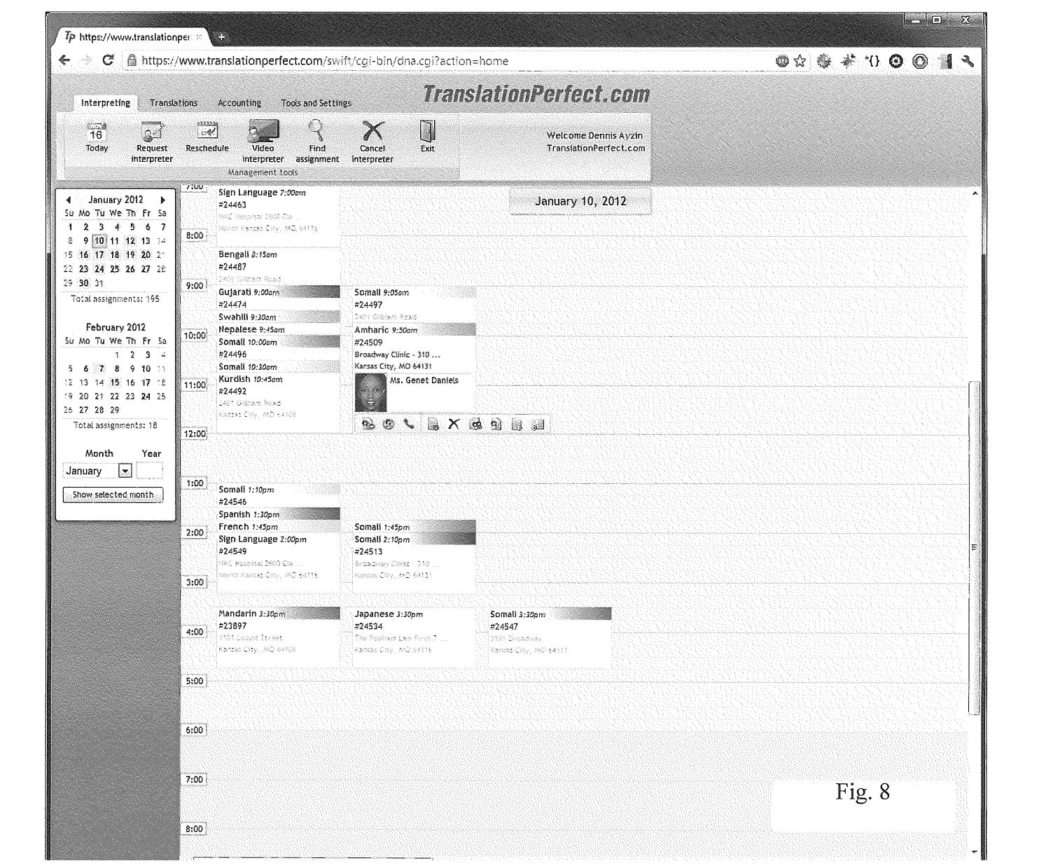

FIG. 8 depicts a calendar entry for an interpretation assignment that has been scheduled with an interpreter. The calendar entry shows details of the assignment and the interpreter and may be linked to other information. For example, the calendar entry may display the place, date, and time of the interpretation assignment, and show the name and a photo of the interpreter scheduled for the assignment. The entry may also include a button that removes the interpreter from the assignment and initiates a new interpreter assignment procedure, a button that is displayed if no interpreter is available for an assignment, and a button that links to the interpreter's profile and/or other information stored in memory.

The entry may also include a button that cancels the interpretation assignment entirely and a button that leads to the Assign Interpreter screen of FIG. 7. Another button links to the agreements executed by the interpreter and another icon links to a time sheet for the assignment. Finally, the calendar entry may include a button that the interpreter may select to upload a time sheet after an assignment is complete.

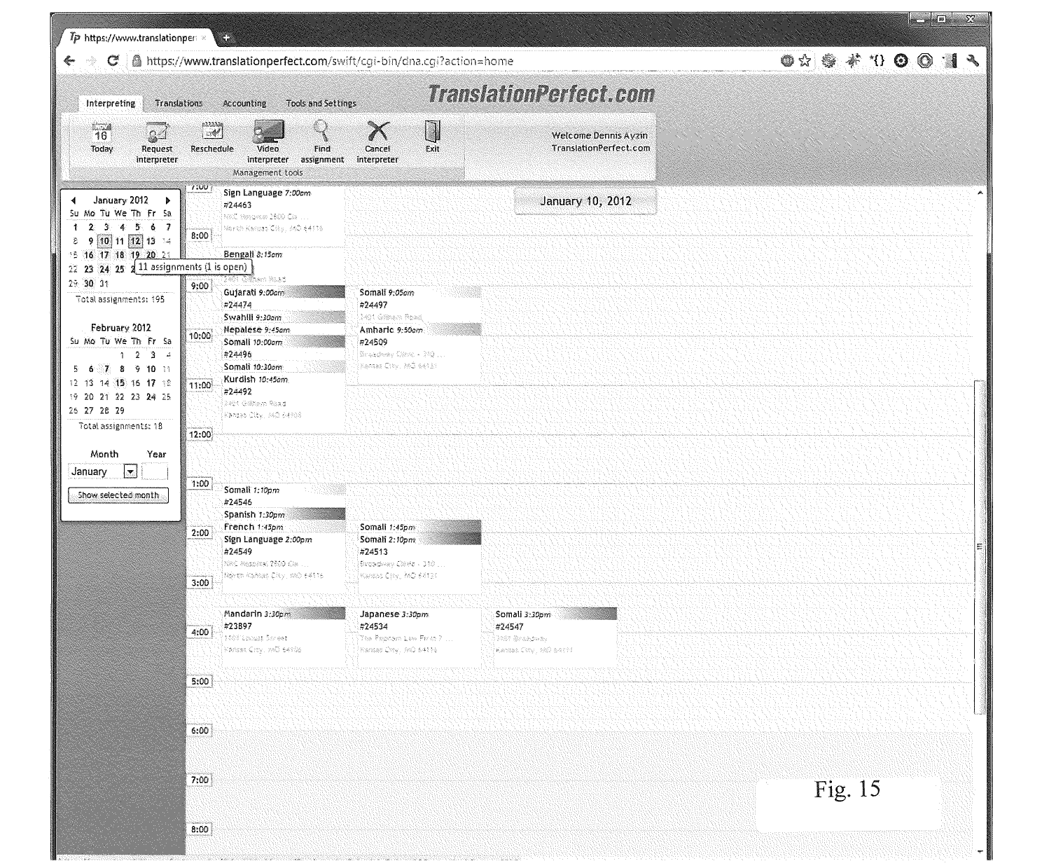

The calendar screens may also provide other useful information for administrators and others. For example, as shown in FIG. 15, the individual days shown on the monthly calendars on the left may be shaded or color-coded to indicate the existence and status of interpretation assignments. In one embodiment, the days with interpretation assignments are bolded or highlighted and the days without assignments are not. Days with interpretation assignments that have been scheduled with an interpreter may also be highlighted or color-coded to distinguish them from assignments without scheduled interpreters. The screen of FIG. 5 may also display the number of interpretation assignments on a given day and indicate how may have scheduled interpreters.

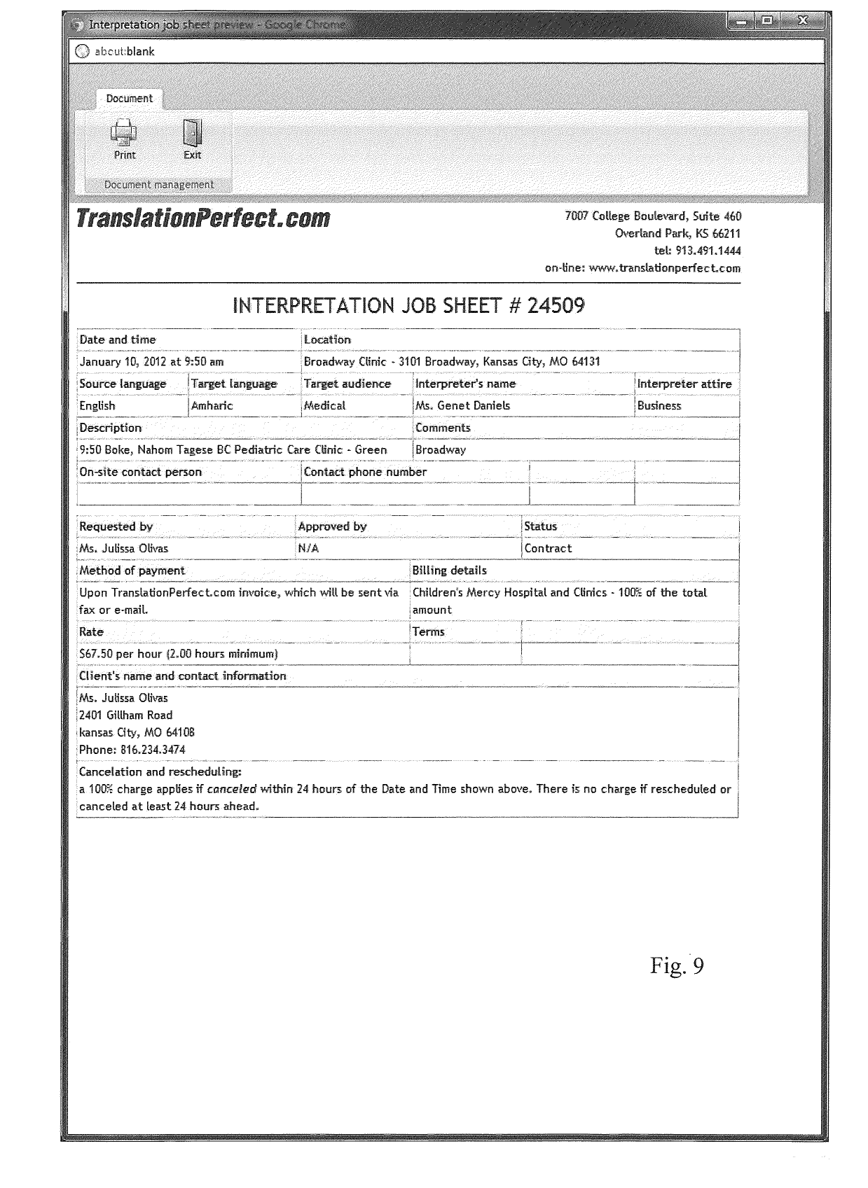

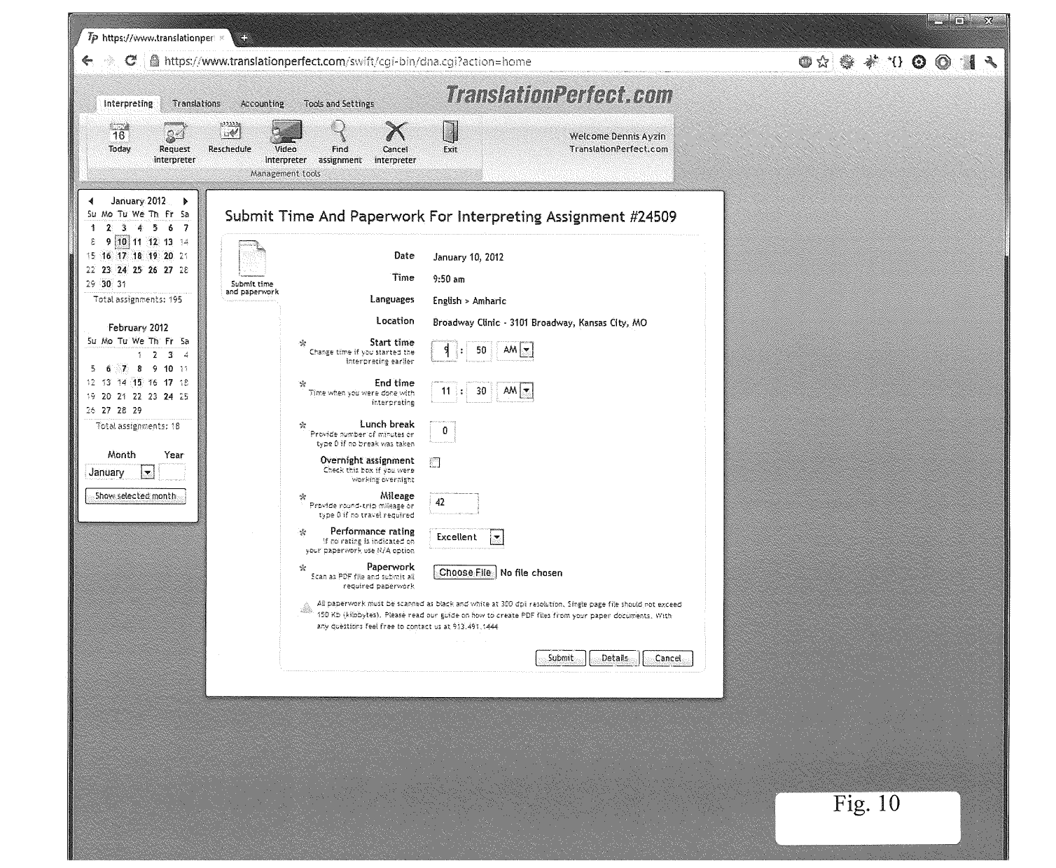

After an interpreter has been identified, selected, and scheduled as described above, the interpreter completes the interpretation assignment in a conventional manner. The interpreter or customer then sends the computer system 12 a message or other indication that the assignment has been completed as shown in box 324. In one embodiment, an interpreter may be required to submit a time sheet or other documents to confirm an assignment has been completed as depicted in box 326. For example, a time sheet such as the one shown in FIG. 9 may be provided to the interpreter when the interpreter accepts an assignment. This can be done as part of the steps in block 314. Once the interpreter completes the interpretation assignment, he or she may be prompted to access the computer system and complete the screen of FIG. 10. This screen prompts the interpreter to scan or otherwise obtain a digital version of the time sheet and submit it to the computer system. The calendar entry may then be updated to include a link to the time sheet as shown in block 328.

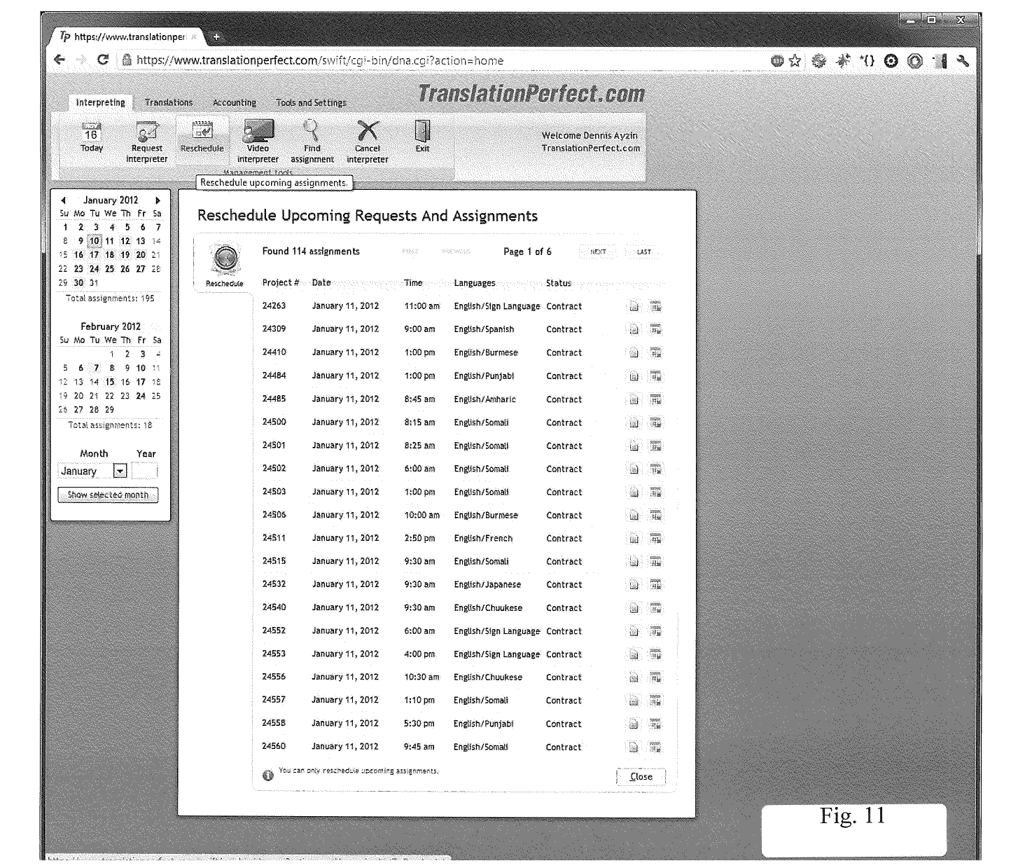

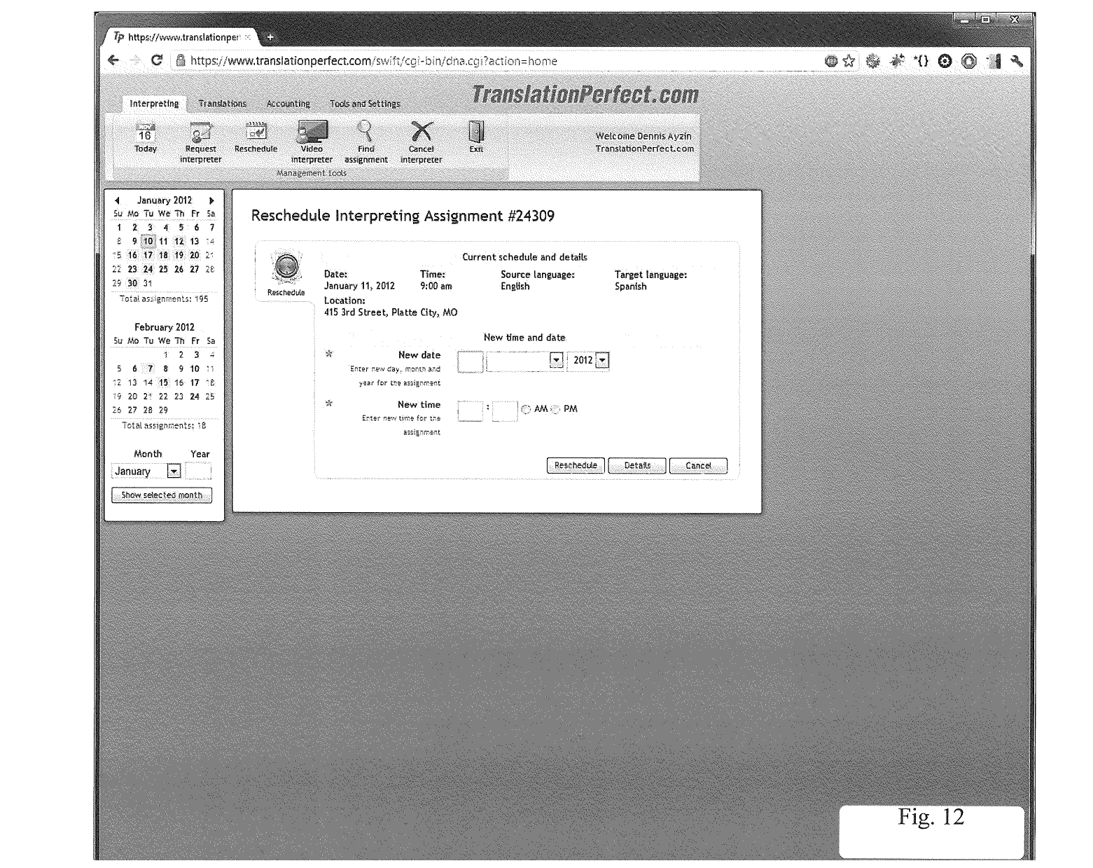

The computer programs of the present invention may also provide other functions and screen displays. For example, the “Reschedule” tab of FIG. 5 may link to the screen of FIG. 11, which lists all upcoming interpretation assignments. The administrator may select an assignment from the list to display the screen of FIG. 12. The screen of FIG. 12 permits the administrator to enter a new date and/or time for an assignment. The administrator may then select a “Reschedule” button which prompts the computer system to send confirmation messages to the interpreter, the customer, and the administrator. If the interpreter indicates he or she is not available at the new date and time, the interpretation assignment is flagged as “unassigned” and the interpreter selection procedures described above must be repeated.

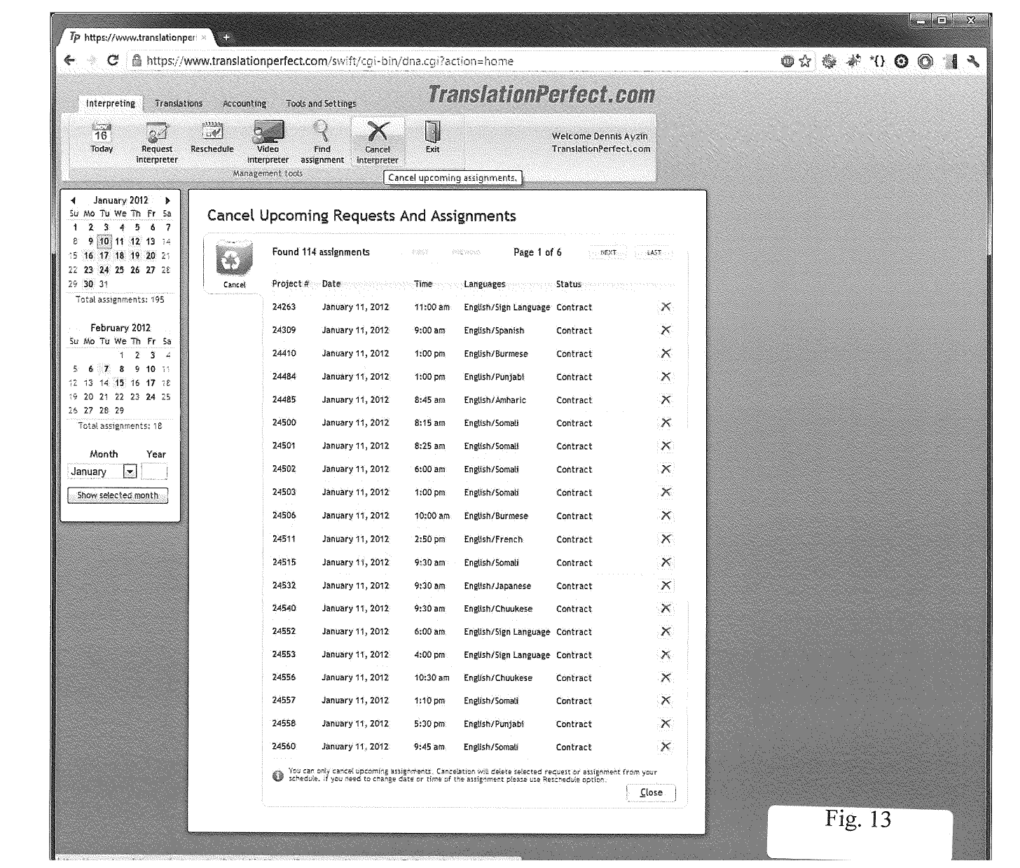

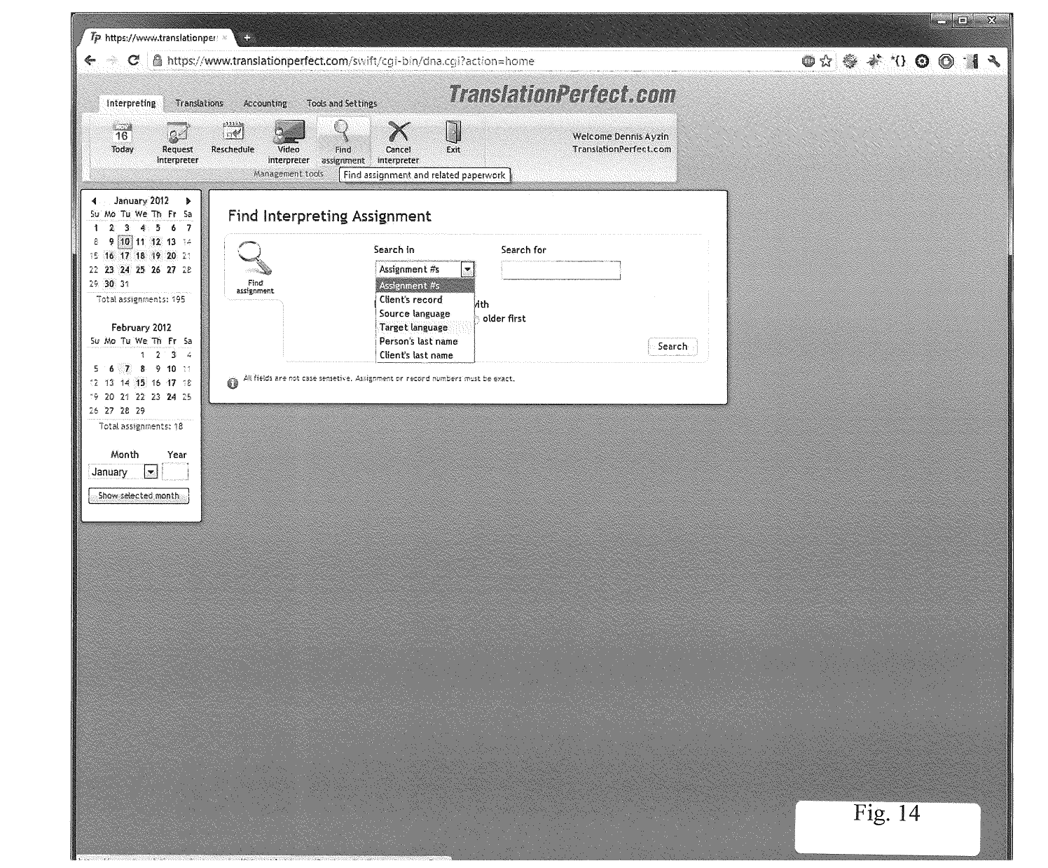

Similarly, the “Cancel Interpreter” tab of FIG. 5 may link to the screen of FIG. 13, which is similar to the screen of FIG. 12 except that it is used to entirely cancel an interpretation assignment. The “Find Assignment” tab on FIG. 5 may link to the screen of FIG. 14. This screen permits an administrator to search for interpretation assignments using several different search fields.

Although the invention has been described with reference to the preferred embodiment illustrated in the attached drawing figures, it is noted that equivalents may be employed and substitutions made herein without departing from the scope of the invention as recited in the claims. For example, although embodiments of the invention may be implemented with the computer and communications equipment described herein, the invention is not limited to this particular computer and communications equipment.

Having thus described the preferred embodiment of the invention, what is claimed as new and desired to be protected by Letters Patent includes the following:

1. A computer-implemented method of scheduling interpreters, the method comprising:
receiving at a computer system information for a number of interpreters;
storing at least some of the interpreter information in memory accessible by the computer system;
receiving at the computer system a request from a customer for an interpreter to perform an interpretation assignment;
searching with the computer system the interpreter information in the memory to identify interpreters who meet a criteria of the request;
providing information about the interpretation assignment to the interpreters who meet the criteria;
receiving at the computer system an availability acknowledgment from at least one of the interpreters;
scheduling the interpretation assignment with one of the interpreters who provides the availability acknowledgment; and
notifying the customer that the interpretation assignment has been scheduled.

2. The method as set forth in claim 1, further comprising:
notifying with the computer system an administrator that the interpretation assignment has been scheduled.

3. The method as set forth in claim 1, further comprising:
providing a calendar accessible on the computer system;
displaying a calendar entry for the interpretation assignment on the calendar; and
linking the calendar entry to at least some of the interpreter information for the interpreter who is scheduled to handle the interpretation assignment.

4. The method as set forth in claim 3, further comprising:
providing an interpretation agreement to the interpreter who is scheduled to handle the interpretation assignment;
receiving and storing an executed copy of the interpretation assignment in memory accessible by the computer system; and
linking the interpretation agreement with the calendar entry on the calendar.

5. The method as set forth in claim 3, further comprising:
receiving from the interpreter who is scheduled to handle the interpretation assignment a time sheet after the interpretation assignment is complete;
storing an executed copy of the time sheet in memory accessible by the computer system; and
linking the time sheet with the calendar entry on the calendar.

6. The method as set forth in claim 3, further comprising:
highlighting on the calendar entries for all interpretation assignments that have not been scheduled with an interpreter.

7. The method as set forth in claim 1, wherein the interpreter information for each interpreter includes the interpreter's name and a list of languages spoken by the interpreter.

8. The method as set forth in claim 1, wherein the request for the interpretation assignment includes a date of the interpretation assignment and an indication of the languages to be interpreted.

9. The method as set forth in claim 1, wherein the criteria of the request includes languages to be interpreted during the interpretation assignment.

10. A non-transitory computer-readable medium for storing a computer program that directs operation of a computer system, the computer program comprising:
a code segment for receiving information for a number of interpreters;
a code segment for storing at least some of the interpreter information in memory accessible by the computer system;
a code segment for receiving a request from a customer for an interpreter to perform an interpretation assignment;
a code segment for searching the interpreter information in the memory to identify interpreters who meet a criteria of the request;
a code segment for providing information about the interpretation assignment to the interpreters who meet the criteria;
a code segment for receiving an availability acknowledgment from at least one of the interpreters;
a code segment for scheduling the interpretation assignment with one of the interpreters who provides the availability acknowledgment; and
a code segment for notifying the customer that the interpretation assignment has been scheduled.

11. The non-transitory computer-readable medium as set forth in claim 10, further comprising:
a code segment for notifying an administrator that the interpretation assignment has been scheduled.

12. The non-transitory computer-readable medium as set forth in claim 10, further comprising:
a code segment for providing a calendar accessible on the computer system;
a code segment for displaying a calendar entry for the interpretation assignment on the calendar; and
a code segment for linking the calendar entry to at least some of the interpreter information for the interpreter who is scheduled to handle the interpretation assignment.

13. The non-transitory computer-readable medium as set forth in claim 12, further comprising:
a code segment for providing an interpretation agreement to the interpreter who is scheduled to handle the interpretation assignment;
a code segment for storing an executed copy of the interpretation assignment in memory accessible by the computer system; and
a code segment for linking the interpretation agreement with the calendar entry on the calendar.

14. The non-transitory computer-readable medium as set forth in claim 12, further comprising:
a code segment for receiving from the interpreter who is scheduled to handle the interpretation assignment a time sheet after the interpretation assignment is complete;
a code segment for storing an executed copy of the time sheet in memory accessible by the computer system; and
a code segment for linking the time sheet with the calendar entry on the calendar.

15. The non-transitory computer-readable medium as set forth in claim 12, further comprising:
a code segment for highlighting on the calendar entries for all interpretation assignments that have not been scheduled with an interpreter.

16. The non-transitory computer-readable medium as set forth in claim 10, wherein the interpreter information for each interpreter includes the interpreter's name and a list of languages spoken by the interpreter.

17. The non-transitory computer-readable medium as set forth in claim 10, wherein the request for the interpretation assignment includes a date of the interpretation assignment and an indication of the languages to be interpreted.

18. The non-transitory computer-readable medium as set forth in claim 10, wherein the criteria of the request includes languages to be interpreted during the interpretation assignment.

19. A computer-implemented method of scheduling interpreters, the method comprising:
receiving at a computer system information for a number of interpreters;
storing at least some of the interpreter information in memory accessible by the computer system;
receiving at the computer system a request from a customer for an interpreter to perform an interpretation assignment;
searching with the computer system the interpreter information in the memory to identify interpreters who meet a criteria of the request;
providing information about the interpretation assignment to the interpreters who meet the criteria;
receiving at the computer system an availability acknowledgment from at least one of the interpreters;
identifying a suitable interpreter from the available interpreters with the computer system;
scheduling the interpretation assignment with the identified interpreter;
notifying the customer that the interpretation assignment has been scheduled;
providing a calendar accessible on the computer system;
displaying a calendar entry for of the interpretation assignment on the calendar; and
linking the calendar entry to at least some of the interpreter information for the interpreter who is scheduled to handle the interpretation assignment.

20. The method as set forth in claim 19, further comprising:
providing an interpretation agreement to the interpreter who is scheduled to handle the interpretation assignment;
storing an executed copy of the interpretation assignment in memory accessible by the computer system; and
linking the interpretation agreement with the calendar entry on the calendar.
